(19) United States
(12) Patent Application Publication
(10) Pub. No.: US 2024/0019079 A1
(43) Pub. Date: Jan. 18, 2024

(54) ELECTRONIC DEVICE POSITIONING STRUCTURE

(71) Applicant: OXTI Pte Ltd, Singapore (SG)

(72) Inventor: Chih-Feng Ho, Singapore (SG)

(21) Appl. No.: 17/867,663

(22) Filed: Jul. 18, 2022

(51) Int. Cl. F16M 13/02; A61L 2/10

(52) U.S. Cl. CPC F16M 13/022; A61L 2/10

(57) ABSTRACT

An electronic device positioning structure includes: an electronic device; and a main body, being bendable, and having a first end and second end, the first end provided with a movable joint slidable relative to the main body, the movable joint in combination with the electronic device, and the second end provided with an accommodating space. Whereby, the main body can be bent into a hook shape for use to hang on an object, and the movable joint can be slid relative to the main body to adjust the relative position of the electronic device 1. For example, the electronic device may be an electronic lamp for sterilization, and the electronic lamp is hung on the chair back to carry out sterilization and disinfection to the chair. Furthermore, the main body can be bent into a straight sheet shape to save space and facilitate transportation, sales and storage.

### (a) TECHNICAL FIELD OF THE INVENTION

The present invention relates to a positioning structure, and more particularly to an electronic device positioning structure.

### (b) DESCRIPTION OF THE PRIOR ART

Chinese Patent No. CN206880873U, titled “height-adjustable ultraviolet germicidal lamp” includes a lamp housing, controller, telescopic pole, counterweight base, reflector and tightening knob, characterized in that the lamp housing is fixed on an elbow installed on the upper side of the telescopic pole through the tightening knob; the middle of the telescopic pole is provided with the controller; the telescopic pole is configured on a sleeve fixed on the counterweight base, the four corners of the lower side of which are respectively installed with a universal wheel; the lamp housing is installed with the reflector, the right and left ends of which is respectively provided with a ultraviolet lamp holder installed with the ultraviolet lamp.

However, the lamp of the above patent is inconvenient in transportation and storage, since the counterweight base and universal wheels provided thereon occupies too much plane space.

### SUMMARY OF THE INVENTION

To overcome the above disadvantages, the present invention proposes an electronic device positioning structure, including: an electronic device; and a main body, being bendable, and having a first end and second end, the first end provided with a movable joint slidable relative to the main body, the movable joint in combination with the electronic device, and the second end provided with an accommodating space,
wherein, one side of the movable joint is provided with a plurality of sharp teeth and a liftable cover.
wherein, the electronic device is an electronic lamp, smart phone, screen, tablet or display panel.
wherein, the movable joint is provided with a groove for the electronic device to be embedded, allowing the movable joint to be in combination with the electronic device.
wherein, the accommodating space of the second end of the main body is provided with an accommodated object, and the accommodated object is a power supply in electric connection with the electronic device.

Whereby, the main body can be bent into a hook shape for use to hang on an object, and the movable joint can be slid relative to the main body to adjust the relative position of the electronic device 1. For example, the electronic device may be an electronic lamp for sterilization, and the electronic lamp is hung on the chair back to carry out sterilization and disinfection to the chair. Furthermore, the combined state of the electronic device and the movable joint can be released, and the main body is then bent into a straight sheet shape to save space and facilitate transportation, sales and storage.

### BRIEF DESCRIPTION OF THE DRAWINGS

FIG. 1 is a perspective view of an electronic device positioning structure of the present invention;

FIG. 2 is an exploded view of the electronic device positioning structure of the present invention;

FIG. 3 is a block chart of the electronic device positioning structure of the present invention;

FIG. 4 is an enlarged perspective view of a movable joint of the present invention;

FIG. 5 is a schematic view of the electronic device positioning structure of the present invention in use;

FIG. 6 is a schematic view of the present invention, where the movable joint is slid relative to the main body;

FIG. 7 is a schematic view of the present invention, where an electronic lamp is controlled to emit light in a wireless way;

FIG. 8 is a schematic view of the present invention, where an electronic lamp is controlled to emit light in a wired way; and

FIG. 9 is a perspective view of the present invention, where the electronic device is a screen.

### DETAILED DESCRIPTION OF THE PREFERRED EMBODIMENTS

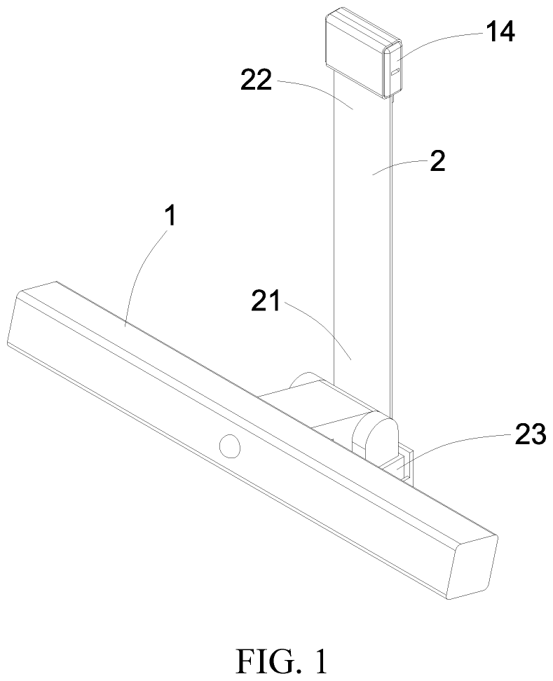

Referring to FIG. 1, an electronic device positioning structure of the present invention includes an electronic device 1 and main body 2.

The electronic device 1 may be chosen from an electronic lamp, smart phone, screen, tablet or display panel, but the present invention is not so limited. The electronic device 1 may be an electronic lamp for sterilization, such as an ultraviolet (UV) radiation lamp or other electronic lamps for sterilization.

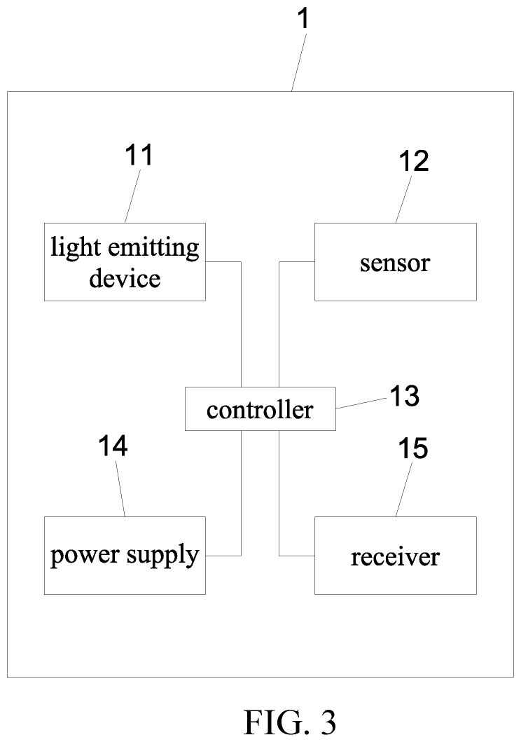

Referring to FIG. 3, specifically, when the electronic device 1 is an electronic lamp for sterilization, the electronic lamp may include a light-emitting device 11, sensor 12, controller 13, power supply 14 and receiver 15, where the light-emitting device 11 may be a light bulb, light string or light-emitting diode (LED), and is used as a light source. The sensor 12 may be a light sensor or infrared sensor to provide a sensing signal. The controller 13 may be a processor or microcontroller adapted to control the light-emitting device 11, and receive the sensing signal emitted from the sensor 12, and further control the light-emitting device 11 according to the sensing signal. The power supply 14 may be a battery or other power supply equipment, used as a power source of the light-emitting device 11. The receiver 15 may be a wireless signal receiver or wired signal receiver, used to receive the wireless signal or wired signal, and transfer the wireless signal or wired signal to the controller 13, allowing the controller 13 to control the light-emitting device 11 according to the wireless signal or wired signal.

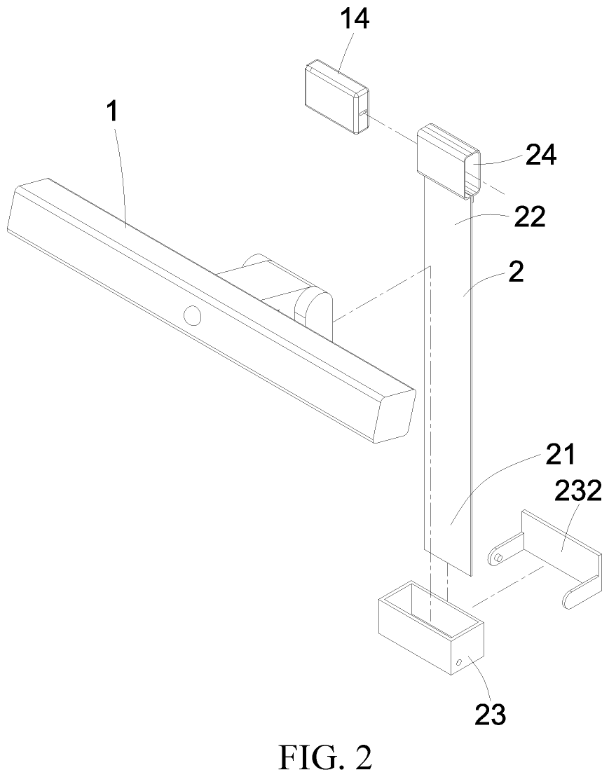

Referring to FIGS. 1 and 2, the main body 2 is in a bendable state, and has a first end 21 and second 22, where the first end 21 is provided with a movable joint 23 that can slide relative to the main body 2, the movable joint 23 being in combination with the electronic device 1, and the second end 22 is provided with an accommodating space 24. Specifically, the body 2 may be made of a flexible material, such as leather, silicone, plastic, cloth or other soft material. The accommodating space 24 of the second end 22 of the main body 2 is provided with an accommodated object, and the accommodated object may be the power supply device 14 electrically connected with the electronic device 1. In addition, the accommodated object may also be other items.

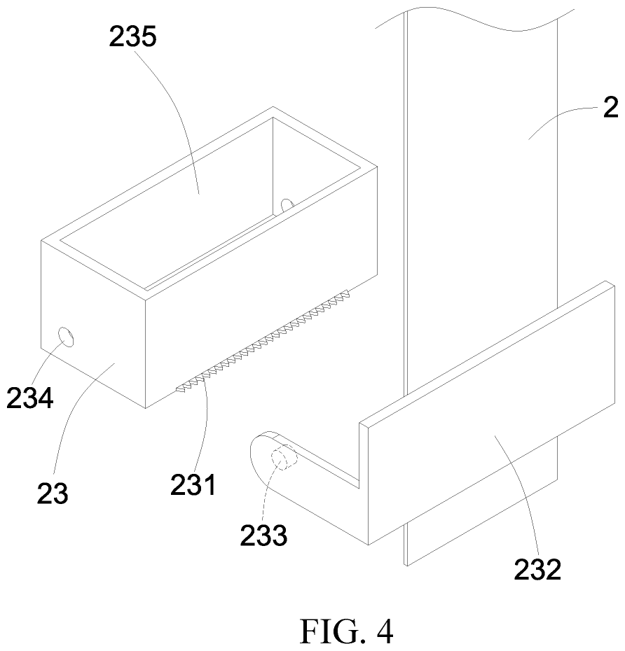

Referring to FIGS. 2 and 4, one side of the movable joint 23 may be provided with a plurality of sharp teeth 231 and a liftable cover 232, where the cover 232 can be rotatably connected to the movable joint 23 through a rotating shaft 233 and shaft hole 234, and at the same time, a gap is formed between the cover 232 and the movable joint 23, where the gap allows the main body 2 to be passed through, making the movable joint 23 slide relative to the main body 2; after the main body 2 is passed through the gap, the cover 232 can be rotated toward the movable joint 23, allowing the cover 232 to be covered on the movable joint 23, and the plurality of sharp teeth to bite the main body 2, preventing the movable joint 23 from falling off the main body 2. The movable joint 23 may be provided with a groove 235 for the electronic device 1 to be embedded, so that the movable joint 23 is combined with the electronic device 1.

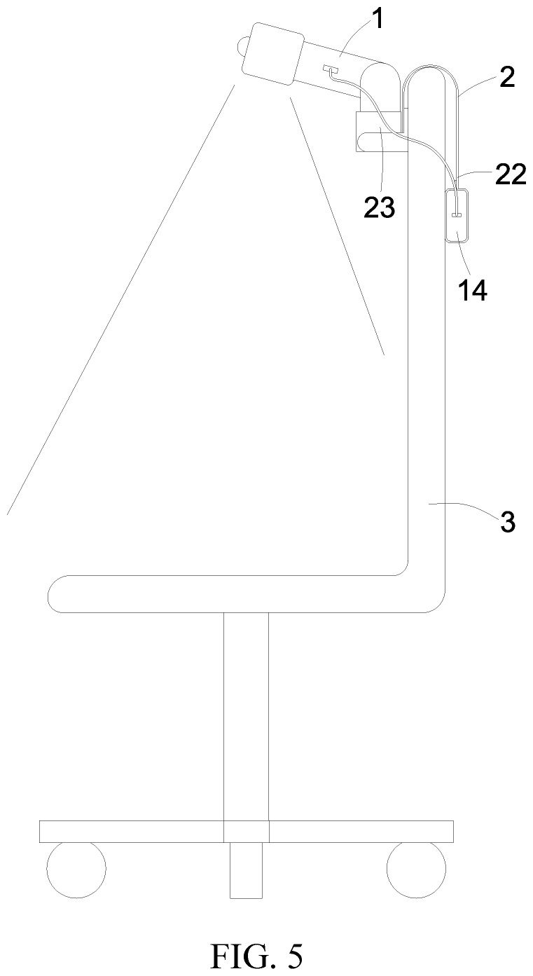

Referring to FIG. 5, when the electronic device positioning structure of the present invention is in use, the main body 2 can be bent into a hook shape for use to hang on an object (such as a chair 3), and the movable joint 23 can be slid relative to the main body 2 to adjust the relative position of the electronic device 1. For example, the electronic device 1 may be an electronic lamp for sterilization, and the electronic lamp is hung on the chair back to carry out sterilization and disinfection to the chair 3. Furthermore, the combined state of the electronic device 1 and the movable joint 23 can be released (the electronic device 1 can be pulled out from the groove 235 of the movable joint 23), and the main body 2 is then bent into a straight sheet shape to save space and facilitate transportation, sales and storage.

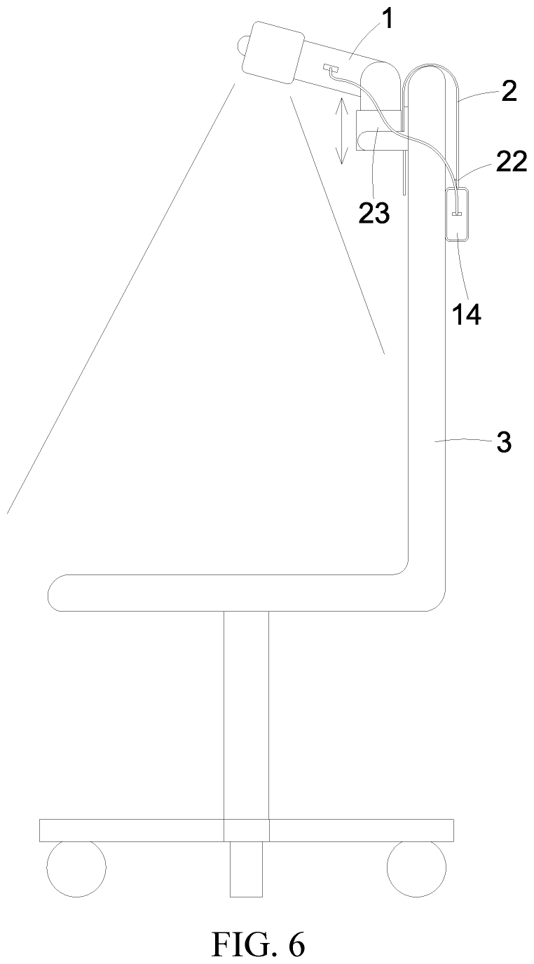

Referring to FIGS. 5 and 6, in the electronic device positioning structure of the present invention, the movable joint 23 is slid relative to the main body 2 to adjust the electronic device 1 to a proper position, for example, the electronic lamp is slid to adjust the position of the required lighting.

Referring to FIGS. 3 and 5, when the electronic device 1 is an electronic lamp for sterilization,

The sensor 12 will continuously detect the surroundings of the chair 3 after the power supply 14 is power on; when sensing somebody approaching the chair 3, the sensor 12 notifies the controller 13, and the controller 13 controls the light-emitting device 11 to stop emitting light to avoid harming the human body; when the sensor 12 senses that there is no one around the chair 3, the sensor 12 notifies the controller 13, and the controller 13 controls the light-emitting device 11 to emit light, so that the chair 3 can be sterilized by irradiating the chair 3 with light emitted by the electronic lamp.

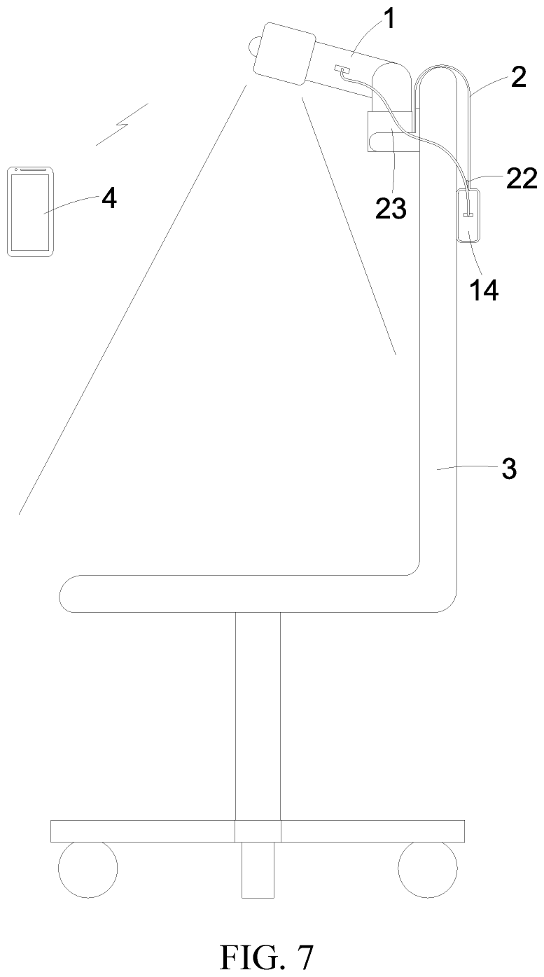

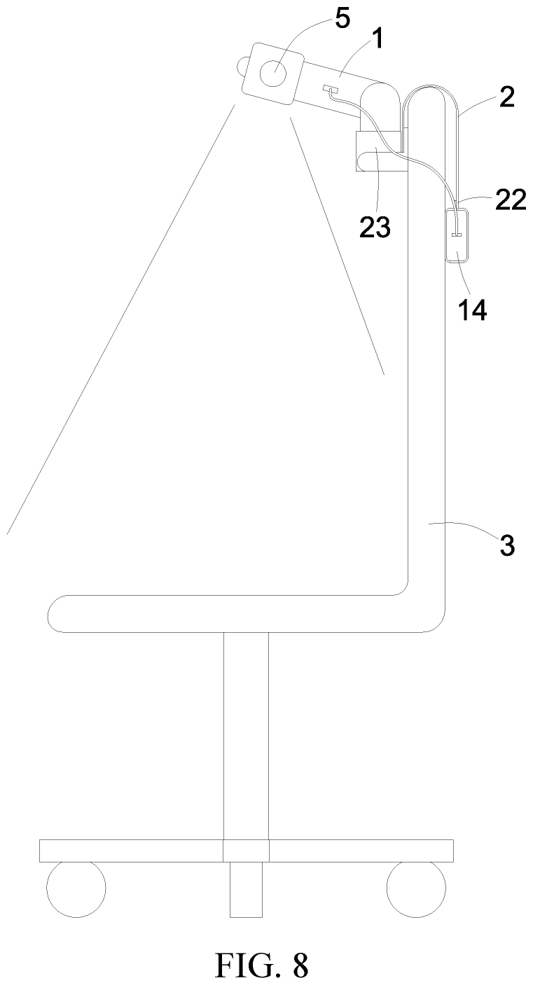

Referring to FIGS. 3 and 7, the present invention can control the electronic lamp to emit light by means of wireless way, and a smart phone 4 or remote controller is then used to provide a wireless signal to the receiver 15, allowing the controller 13 to control the light-emitting device 11 to emit light according to the wireless signal. Referring to FIGS. 3 and 8, the present invention can also control the electronic lamp to emit light by means of wired way, i.e., the electronic lamp is provided with a wired switch 5 in electric connection with the receiver 15, and the wired switch 5 provides a wired signal, and the receiver 15 receives the wired signal and then transfers it to the controller 13, allowing the controller 13 to control the light-emitting device 11 to emit light according to the wired signal.

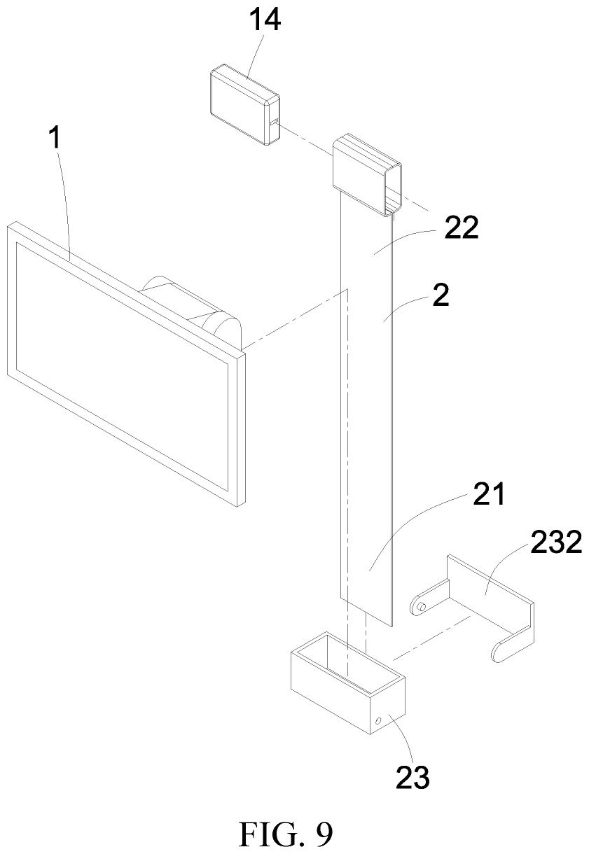

Referring to FIG. 9, the electronic device 1 may also be a screen.

I claim:

1. An electronic device positioning structure, comprising:
an electronic device; and
a main body, being bendable, and having a first end and second end, said first end provided with a movable joint slidable relative to said main body, said movable joint in combination with said electronic device, and said second end provided with an accommodating space.

2. The structure according to claim 1, wherein one side of said movable joint is provided with a plurality of sharp teeth and a liftable cover.

3. The structure according to claim 1, wherein said electronic device is an electronic lamp, smart phone, screen, tablet or display panel.

4. The structure according to claim 1, wherein said movable joint is provided with a groove for said electronic device to be embedded, allowing said movable joint to be in combination with said electronic device.

5. The structure according to claim 1, wherein said accommodating space of said second end of said main body is provided with an accommodated object, and said accommodated object is a power supply in electric connection with said electronic device.
